(19) United States
(12) Patent Application Publication
(10) Pub. No.: US 2025/0082713 A1
(43) Pub. Date: Mar. 13, 2025

(54) Active ingredient groups and their key target combinations for the treatment of UC

(71) Applicant: Centre for Chinese Herbal Medicine Drug Development Limited, Hong Kong (CN)

(72) Inventors: Zhaoxiang Bian, Hong Kong (CN); Lin Zhu, Hong Kong (CN); Ka Wing Cheng, Hong Kong (CN); Hor Yue Tan, Hong Kong (CN); Jingchun Shi, Hong Kong (CN); Chunhua Huang, Hong Kong (CN); Chengyuan Lin, Hong Kong (CN)

(21) Appl. No.: 18/462,749

(22) Filed: Sep. 7, 2023

(51) Int. Cl. A61K 36/9066; A61K 31/12; A61K 31/365; A61K 31/4375; A61K 31/7034; A61K 31/704; A61K 36/076; A61K 36/284; A61K 36/344; A61K 36/40; A61K 36/484; A61K 36/718; A61K 36/74; A61K 36/898; A61P 1/04

(52) U.S. Cl. CPC A61K 36/9066; A61K 31/12; A61K 31/365; A61K 31/4375; A61K 31/7034; A61K 31/704; A61K 36/076; A61K 36/284; A61K 36/344; A61K 36/40; A61K 36/484; A61K 36/718; A61K 36/74; A61K 36/898; A61P 1/04

(57) ABSTRACT

The present invention belongs to the field of medical technology and specifically relates to an active ingredient group for treating UC. An active ingredient group for treating UC, characterized in that it uses an active ingredient group to act on key target combination of UC, and prevents and treats UC by regulating tryptophan metabolism, glycerolphospholipid metabolism, and linoleic acid metabolism. The active ingredient group for treating UC provided in this application can effectively prevent and treat UC by regulating tryptophan metabolism, glycerolphospholipid metabolism and linoleic acid metabolism by establishing strong combination between the active ingredient group and the key target combination of UC. This application also discloses the biomarkers for the diagnosis and treatment of UC.

### CROSS-REFERENCE TO RELATED APPLICATIONS

This non-provisional application claims priority under 35 U.S.C. § 119(a) on Patent Application No. 202310921375.0 filed in China on Jul. 26, 2023, the entire contents of which are hereby incorporated by reference.

### TECHNICAL FIELD

The present invention belongs to the field of medical technology, Especially involving An active ingredient group for treating UC.

### BACKGROUND OF THE INVENTION

The inventor of this application submitted a US invention patent application on Feb. 11, 2023, titled A Pharmaceutical Composition For Preventing And Treating Inflammatory Bowel Disease, with patent application Ser. No. 18/167,842. In the inventor's previous patent application (application number 18/167842), the drug combination and clinical medical effects of BUCD2103 were recorded in detail. The publicly available drug combination BUCD2103 is referred to as CDD-2103 in this application.

Ulcerative Colitis (UC) is a chronic recurrent gastrointestinal disease. The standard treatment for UC remission is often unsatisfactory. CDD-2103 is a traditional Chinese medicine preparation that has been proven to be effective in treating UC. In fact, our pharmacodynamic studies have shown that CDD-2103 alleviates ulcer symptoms and colon damage in DSS induced chronic colitis mouse models. However, the mechanism of this effect is not yet clear, and further research is needed on the target and active ingredients of CDD-2103 to improve the accuracy and effectiveness of UC disease diagnosis and treatment, and provide further theoretical support for the medical treatment and prevention of UC.

### SUMMARY OF THE INVENTION

In view of this, this application provides an active ingredient group for treating UC to solve some or all of the technical problems defined in the background technical section of this application.

The active ingredient group for treating UC provided in this application to address its technical issues are:

An active ingredient group for treating UC, characterized by:
the active ingredient group includes curcumin, berberine, atractylolactone III, glycyrrhizin and militarine.

Preferably, the active ingredient group can act on the key target combination of UC, which includes MA0A, MAPK14, AHR, PTGS2, PLA2G1B, and ALOX5.

Preferably, the active ingredient group can regulate tryptophan metabolism, glycerolphospholipid metabolism, and linoleic acid metabolism.

Preferably, there is a strong binding between the active ingredient group and the key target combination of UC.

Preferably, the biomarkers for treating UC using the active ingredient group are KA, PC, and AA.

Preferably, the active ingredient group comes from CDD-2103.

Preferably, CDD-2103 comprises:
Codonopsis Radix,
Bran-processed Atractylodis Macrocephalae Rhizoma,
Poria,
Wine-processed Cori Fructus,
Coptidis Rhizoma,
Curcumae Longae Rhizoma,
Paederiae Scandentis Herba et Radix,
Bletillae Rhizoma,
Honey-processed Radix Glycyrrhizae.

### Benefits of this Application

The active ingredient group for treating UC provided in this application can effectively prevent and treat UC by regulating tryptophan metabolism, glycerophospholipid metabolism and linoleic acid metabolism by establishing strong combination between the active ingredient group and the key target combination of UC. At the same time, the biomarkers for the diagnosis and treatment of UC have also been disclosed.

The following is a detailed introduction to the technical solution and effects of this application, combined with the accompanying drawings and specific implementation methods of the specification.

### BRIEF DESCRIPTION OF DRAWINGS

FIG. 1, CDD-2103 exhibited protective effects in DSS-induced chronic colitis mice. (A-B) CDD-2103 inhibited body weight loss and decreased DAI score in DSS-treated mice. (C) CDD-2103 reversed colon shortening in DSS-induced colitis mice. (D) Representative images of H&E staining (magnification, 4×): (a) CTR group; (b) DSS group; (c) SASP group; (d) CDD-2103 5.8 g/kg group; (e) CDD-2103 8.8 g/kg group; (f) CDD-2103 11.7 g/kg group. Data are presented as the mean±S.D. (n=12). *, #: p<0.05, and **, ##: p<0.01; #: comparison between CTR group and DSS group; * comparisons between CDD-2103/SASP and DSS group.

FIG. 2, PLS-DA score plots of the metabolic profiles in serum and fecal samples from the six groups of mice via the positive and negative mass spectrometry scan modes.

FIG. 3, The heat map of differential metabolites in feces (A) and serum (B) samples among control (CTR), DSS, low- (L), medium- (M) and high- (H) doses of CDD-2103 and SASP group. The enriched metabolic pathways in feces (C) and serum (D) samples.

FIG. 4, CDD-2103 modulated tryptophan metabolism in DSS-treated mice. (A) Fecal and (B) serum tryptophan metabolites levels in CTR, DSS, SASP, and CDD-2103 groups. Data are presented as the mean±S.D. (n=12). *, #: p<0.05, and **, ##: p<0.01; #: comparison between CTR group and DSS group; * comparisons between CDD-2103/SASP and DSS group.

FIG. 5, Venn diagram of CDD-2103 compounds identified by serum-feces pharmacochemistry analysis.

FIG. 6, Network pharmacology analysis of CDD-2103 for treating UC. (A) The Venn diagram showed the overlapping genes of CDD-2103 and UC. (B) Core genes were extracted from the PPI network of the overlapping genes. The bigger size and the denser color of the node represented the more important targets. (C) GO enrichment analysis of the biological function of the significant genes. (D) KEGG pathway enrichment of significant genes of CDD-2103 against UC. (E) Herb-compound-gene-pathway-disease network, the circles with different color represented CDD-2103 compounds from different herbs, the yellow circles represented genes, the ren V represented pathways. The bigger size represented the larger degree. SY1: morroniside; SY2: loganin; HL1: magnoflorine; HL2: jatrorrhizine-3-O-beta-D-glucuronide; HL3: berberrubine; HL4: coptisine; HL5: columbamine; HL6: epiberberine; HL7: jatrorrhizine; HL8: berberine; HL9: palmatine; GC1: liquiritin; GC2: methylcyclopentane; GC3: glycyrrhizic acid; BJ1: α-Isobutylmalic acid; BJ2: militarine; FL1: poricoic acid DM; BZ1: atractylenolide III; JH1: bisdemethoxycurcumin; JH2: demethoxycurcumin; JH3: curcumin.

FIG. 7, The integrated network of compound-reaction-enzyme-gene of the metabolites (red node) and genes (blue node), the yellow node indicating the key metabolites and genes.

FIG. 8, The integrated network of “herb-compound-target-pathway-metabolite-disease”, the nodes in each column are herbs (CDD-2103), compounds, targets, pathways, metabolites, and disease (UC) from left to right.

FIG. 9, Detailed molecular docking results of the strongest binding affinity pair (kJ/mol). Cartoon representation for the protein target and the space-filling for the small molecular compound.

FIG. 10, Summary depiction of the putative action mechanisms of CDD-2103 in UC remission.

### DETAILED DESCRIPTION OF THE INVENTION

The specific validation experiments and theoretical studies using CDD-2103 as a UC treatment drug will provide a detailed explanation of the active ingredient group for treating UC disclosed in this application.

### 1. Material and Methods

### 1.1 Reagents

Dextran sulfate sodium (DSS; MW: 36-50 kDa) was bought from MP Biomedicals (USA). Sulfasalazine (SASP) was acquired from Europharm Laboratoires Company Limited (China). The hematoxylin-eosin (H&E) staining solution was obtained from Sigma-Aldrich (USA). The reference standards of berberine hydrochloride, epiberberine, coptisine chloride, palmatine, morroniside, loganin, and curcumin were purchased from Meilunbio (China). Militarine, glycyrrhizin ammonium salt, jatrorrhizine, columbamine, liquiritin, demethoxycurcumin, bisdemethoxycurcumin, berburrubine, and magnoflorine were purchased from CFW Laboratories (China). The purity of all standards was ≥98.0% (HPLC).

### 1.2 Preparation of CDD-2103

The powdered extract of CDD-2103 (batch no. J-220606-01) was provided by Beijing Increasepharm Corporation Limited (China). All herbs were authenticated by Pony Testing International Group (China) according to the Chinese Pharmacopoeia (version 2020) or Chinese Herbal Medicine Quality Standard (Hebei Province, version 2018). Briefly, HL, JH, and SY were extracted together with 8-fold 70% ethanol for 1 h three times, under reflux, and another six herbs in CDD-2103 were extracted together with water. The extracts were combined, filtered, and then concentrated by rotary evaporation. The freeze-dried powder was then stored at −20° C. until further use.

### 1.3 DSS-Induced Chronic Colitis Mice Model

The animal experiment was approved by the Committee on the Use of Human and Animal Subjects in Teaching and Research of Hong Kong Baptist University (REC/19-20/0301). Male C57BL6J mice aged 6-8 weeks were purchased from The Chinese University of Hong Kong (China). The mice were kept at a controlled temperature with a 12 h light/dark cycle with free access to water and food. On Day 13, the mice were divided into six groups (n=12/group) as follows: CTR, control group; DSS, group administered with DSS; SASP, group administered with sulfasalazine and DSS; and 3 groups one each administered with low (5.8 g/kg), medium (8.8 g/kg), and high (11.7 g/kg) of CDD-2103 and DSS. Mice in DSS, SASP group and CDD-2103 groups with different dosages were subjected to three cycles of 1.8% DSS to induce chronic colitis during the 49-day experimental period.

The CDD-2103 and SASP groups were orally administered with the CDD-2103 extract and SASP (500 mg/kg), respectively, once per day starting at Day 13. The dosages from the low, medium, and high CDD-2103 groups were based on the clinical effective dosage of CDD-2103 (0.8 g raw herbs/kg/day) experienced in UC patients. They were, 5.8, 8.8, and 11.7 g of raw herbs/kg/day, respectively, (equivalent to 0.8, 1.1 and 1.5 g of raw herbs/kg/day respectively of clinical dosage). The CTR and DSS groups were orally administered with an equal volume of water as in SASP and CDD-2103 groups. On Day 48, fecal samples of all groups were collected and kept at −80° C. until further use. On Day 49, mice were sacrificed, and serum and colon samples were collected.

### 1.4 Measurement of Disease Activity Index and Colon Length

The severity of colitis in mice was scored according to weight loss, the scoring of bloody stool, and stool consistency as reported previously with a minor adjustment. Body weight change was calculated as the difference between the initial body weight on Day 1, and rated as follows: 0, <1% no loss; 1, 1-5% loss; 2, >5-10% loss; 3, >10-15% loss; 4, >15% loss. Hemoccult SENSA kits (USA) were used for bloody stool detection. Results were rated as follows: 0, no blood (brown); 1, some bleeding high in the gastrointestinal tract (slightly blue); 2, significant bleeding high in the gastrointestinal tract (blue); 3, slight bleeding (slightly red); 4, significant bleeding (red). Stool consistency was rated as follows: 0, normal; 1, soft but formed; 2, soft; 3, very soft and wet; 4, watery. Colon length was determined by measuring from the ileocecal junction to the anus.

### 1.5 Histopathological Analysis

After mice sacrifice, distal colons were rinsed and soaked in 4% paraformaldehyde at 4° C. overnight. Colonic tissues were sequentially dehydrated and finally embedded in paraffin blocks. Tissues were segmented into 4 m slices and stained (H&E) for the observation of colonic injuries.

### 1.6 Metabolomics

The freeze-dried feces were extracted with 30-fold volume of 80% methanol (m/v), then mixed with steel beads and processed with Tissue Lyser to obtain a fecal slurry. After incubation at −80° C. for 4 hours, the homogenate was centrifuged (18,000×g, 4° C., 10 min) and the supernatant was collected. Serum was extracted with a 4-fold volume of methanol; the mixture was then vortexed for 1 min and centrifuged (18,000×g, 4° C., 10 min). The supernatant was dried at low temperature under vacuum. The dried samples were reconstituted with equal volume of 50% methanol, vortexed for 5 min, and centrifuged (18,000×g, 4° C., 10 min). The supernatant was used for ultra-high performance liquid chromatography-quadrupole time-of-flight mass spectrometry (UPLC-Q-TOF/MS) analysis. A combined quality control (QC) sample was prepared by mixing equal amount of each sample.

An Agilent 1290 Infinity II UPLC coupled with a 6546 Q-TOF/MS system was used to obtain the fecal and serum metabolomics profiles. A 100 mm×2.1 mm Acquity UPLC HSS C18 1.7 m column was used for analysis. The chromatographic conditions were as follows: mobile phase A, water with 0.1% formic acid; mobile phase B, acetonitrile with 0.1% formic acid; a linear gradient, 0-1 min (1% B), 1-12.5 min (1-100% B), 12.5-14.5 min (100% B), 14.5-14.7 min (100-1% B), 14.7-17.7 min (1% B). MS analysis was carried out in both positive and negative ion modes. The electrospray ionization (ESI) source had the following parameters: gas temperature, 300° C.; drying gas, 8 L/min; nebulizer, 45 psi; sheath gas temperature, 350° C.; sheath gas flow, 8 L/min; voltage of capillary, 3.0 kV. MS1 full scan range was m/z 80-1000; MS2 full scan method was as follows: full scan range: m/z 40-1000; collision energy was set at 10, 20, and 40 eV.

The acquired MS1 raw data was converted to mzXML format by using MSConvert GUI software from the ProteoWizard toolset. The intensities were corrected for batch effect and signal drift by fitting a locally quadratic (loess) regression model to the median intensity of combined QC samples. For multivariate statistical analysis, supervised partial least squares discrimination analysis (PLS-DA) was processed with SIMCA-P software. The features with fold change (FC)>1.2 or <0.8, and p-value<0.05 were considered to be potentially differential compounds. Identification of these compounds was first performed by comparing the accurate MS and MS/MS (ppm<5) with Human Metabolome Database (HMDB). Some of them was verified by comparison with authentic standards. Heat maps were created using the heatmap package in R. Metabolic pathway analysis was enriched by MetaboAnalyst 5.0. For the quantitative analysis of tryptophan metabolites, the sample preparation method was the same as above, using the quantification instrument and method as described in a previous report.

### 1.7 Serum-Feces Pharmacochemistry Studies in Rats

Sprague Dawley rats (200±20 g) were purchased from The Chinese University of Hong Kong (China). Ten male rats were randomly divided into two groups (n=5). After overnight fasting, they were orally administered with 4.7 g raw herbs/kg of CDD-2103 (equivalent to 0.8 g of raw herbs/kg/day of clinical dose used in human). Serum was collected from one group, and feces were collected from the second group. Before administered with CDD-2103, feces, and blood samples were collected from each rat for the self-control study. At 0.25, 0.5, 1.0, and 2.0 h after oral administration of CDD-2103, blood samples (0.2 mL) of rats were collected from the ophthalmic veins. Blood was centrifuged (4000×g, 4° C., 10 min); the supernatant was collected and stored at −80° C. until analysis. At 8.0 and 12.0 h after administration, fecal samples were collected and were directly stored at −80° C. until further use.

### 1.8 Serum-Feces Component Analysis of CDD-2103

For serum analysis, a 5-fold volume of methanol was added to each sample; it was vortexed for 30 s, centrifuged (18,000×g, 4° C., 10 min), and then collected the supernatant. Fecal samples were weight, extracted with 50-fold volume of methanol, and subjected to ultrasonic 30 min, then centrifuged (18,000×g, 4° C., 10 min). The supernatant was used for UPLC-Q-TOF/MS analysis. For phytochemical analysis of both serum and feces samples, the gradients were set as 5-10% B for 0-2 min, 10-15% B for 2-8 min, 15-17% B for 8-10 min, 17-20% B for 10-16 min, 20-50% B for 16-21 min, 50-54% B for 21-24 min, 54-100% B for 24-25 min, 100% B for 25-26 min, and 100-5% B for 26-28 min. The compound database of CDD-2103 was constructed according to the HERB database and the literature. Components in serum and feces were identified based on the accurate m/z, fragmentation data, or by comparison with reference standards.

### 1.9 Network Pharmacology Studies

The HERB database was used to manually search for the molecular targets of the components (Cx) identified from the serum and feces samples of rats administered with CDD-2103. The drug similarity search tool in Therapeutic Targets Database (TTD) was used to recognize drugs' similarity with Cx. Only drugs with a high structural similarity score (>0.70, described as “moderately similar” to “very similar”) were selected. The therapeutic targets of the recognized drugs were also considered to be the potential targets of Cx. The biological targets of UC were collected by searching on the keywords “ulcerative colitis”, “gut immune modulation” and “colon mucosa protection” in GeneCards. The intersection genes of Cx with disease genes were constructed by a Venn diagram. Protein-protein interactions (PPI) of the intersection targets were constructed in a STRING database. We considered the overlapping targets to be therapeutic targets for CDD-2103 in the treatment of UC. Functional enrichment analysis of the intersection genes was carried out by DAVID database. Cytoscape 3.7.2 was utilized to create the herb-compound-gene-pathway-disease network.

### 1.10 Integrated Analysis of Metabolomics and Network Pharmacology

To reveal the altered key metabolites, related pathways, targets and active compounds, integrated networks were constructed, and analyzed. First, the differential metabolites identified by metabolomics and the predicted genes predicted by network pharmacology were imported into the Cytoscape equipped with MetScape to produce a compound-reaction-enzyme-gene network. This construction depicted the interactions among the metabolites, pathways, enzymes, and genes for further analysis. Second, the herbs, active compounds, genes, pathways, and metabolites were imported into Cytoscape to obtain the network. Third, the key metabolites, genes and active compounds were recognized in the two integrated networks.

### 1.11 Molecular Docking

Docking of the key active compounds and genes was explored using CB-Dock, an online molecular docking tool. PDP files of receptors were downloaded from the RCSB Protein Data Bank, and the active ingredients SDF files were downloaded from the PubChem database. All the prepared files were uploaded into CB-Dock. After determining the docking pocket coordinates, molecular docking and conformational scoring were performed using CB-dock. The lower the Vina scores, the more stable is the ligand binding to the receptor; thus, Vina scores were, used for preliminary evaluation of the binding activity of the compounds to the targets.

### 1.12 Statistical Analysis

Statistical analysis of t-test, and one-way ANOVA were performed using GraphPad Prism 8.0 (USA). A p-value<0.05 was considered statistically significant.

### 2. Results

### 2.1. CDD-2103 Treatment Alleviates DSS-Induced Chronic Colitis in Mice

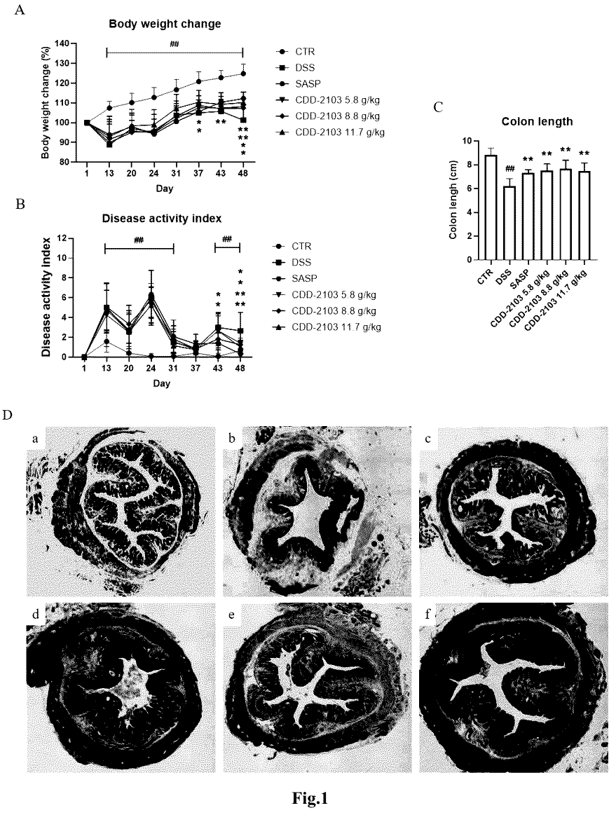

As shown in FIGS. 1A and 1B, since day 13, compared with CTR mice, DSS-treated mice had a pronounced decline in body weight, as well as significant raised in scores of DAI. Administration of CDD-2103 and SASP remarkably reversed body weight loss and decreased scores for DAI in colitis mice at the later period of DSS modeling. Furthermore, compared to the CTR mice, DSS treatment resulted in a remarkable reduction of colon length. Notably, CDD-2103 and SASP treatment improved this DSS-induced chronic colon shortening (FIG. 1C). In DSS-treated mice, the bloody stool and diarrhea were attributed to colonic inflammation and damage in the intestinal wall. As shown in FIG. 1D, severe inflammatory cell infiltration and crypt destruction were observed in the histological sections of colitis mice. Both CDD-2103 and SASP could protect DSS-treated mice from histopathological damage.

### 2.2. Metabolomics Profiling

A total of 17,735 features were determined in all the fecal samples and 7,702 features in all the serum samples. The stability and repeatability of metabolomics were evaluated by QC samples. 96.5% and 96.4% of features in fecal and serum samples, respectively, had an RSD %<30%. The total ion chromatograms (TICs) showed that QC samples behaved stably during the process. These data suggested the high stability of the instrument and the repeatability of the metabolomics method.

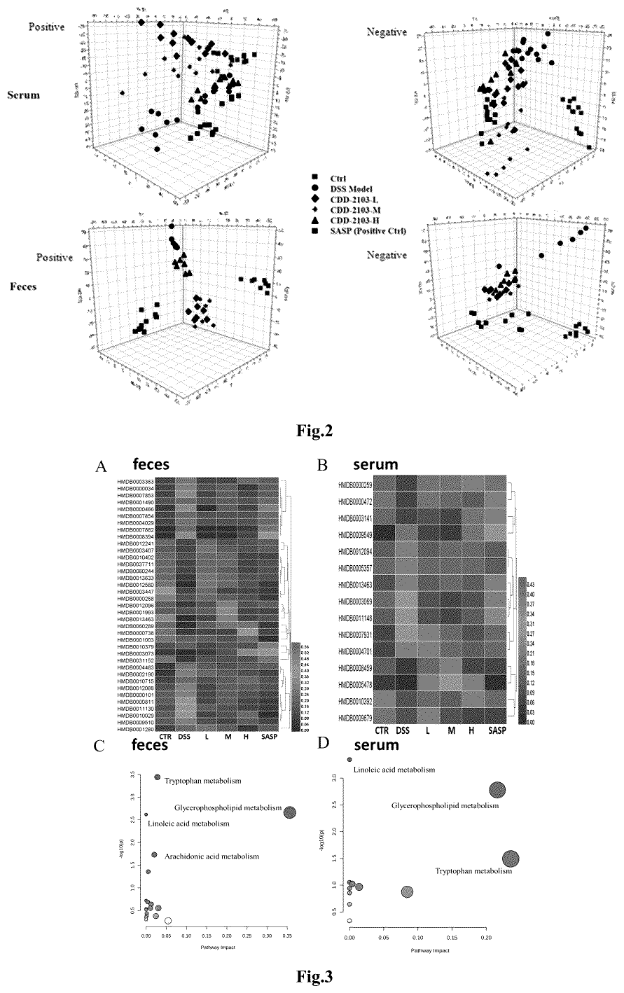

To further investigate the differences among the six groups of mice, we performed PLS-DA analysis. As shown in FIG. 2, PLS-DA displayed that the samples from the same group clustered together and were distinct from samples from other groups, indicating consistency between metabolic variation and phenotype. The parameters of R2Y and Q2 in PLS-DA of fecal samples were 0.978, 0.791 and 0.917, 0.786 in positive and negative ion modes, respectively. These parameters in PLS-DA of serum samples were 0.909, 0.651 and 0.971, 0.750 in positive and negative ion modes, respectively. These results indicated that the DSS inducement and CDD-2103 treatment caused obvious metabolic variations in feces and serum.

### 2.3. Identification of Differential Metabolites and Pathway Analysis

Based on FC>1.2 and p<0.05, according to HMDB, 37 and 15 metabolites were identified in feces and serum, respectively (Tables 1 and 2). To visualize the variation in metabolites among the six groups, we plotted heat maps. FIGS. 3A and 3B showed that the metabolites were changed in the DSS model group and were restored in the CDD-2103 groups (low, medium, and high doses) in serum and feces samples, indicating that CDD-2103 treatment could reverse metabolic perturbation.

TABLE 1
The detailed information of differential metabolites in fecal samples.

| RT (min) | Precursor MS | MS/MS | Compound name | HMDB ID |
|---|---|---|---|---|
| 0.9 | 195.0480 | 123.0087 | Pectic acid | HMDB0003363 |
| 1.4 | 134.0472 | 104.0500 | Adenine | HMDB0000034 |
| 1.8 | 157.0507 | 227.2017 | Isopropylmaleic acid | HMDB0012241 |
| 2.1 | 445.1876 | 152.9958 | Estrone glucuronide | HMDB0004483 |
| 2.8 | 136.0745 | 96.9696 | 2-Phenylacetamide | HMDB0010715 |
| 2.8 | 252.1087 | 205.1961 | Deoxyadenosine | HMDB0000101 |
| 2.9 | 185.0810 | 295.1698 | Vanylglycol | HMDB0001490 |
| 3.5 | 140.0353 | 96.9696 | 2-Aminomuconic acid semialdehyde | HMDB0001280 |
| 3.9 | 130.0661 | 71.0133 | 3-Methylindole | HMDB0000466 |
| 5.0 | 568.3362 | 327.2329 | LysoPC (22:5(4Z, 7Z, 10Z, 13Z, 16Z)/0:0) | HMDB0010402 |
| 5.5 | 466.2921 | 132.0454 | LysoPC (14:0/0:0) | HMDB0010379 |
| 5.6 | 170.0591 | 90.0343 | Phosphodimethylethanolamine | HMDB0060244 |
| 5.7 | 160.0405 | 174.0415 | Quinoline-4,8-diol | HMDB0060289 |
| 5.7 | 116.0508 | 277.2173 | Indole | HMDB0000738 |
| 6.1 | 162.0903 | 98.9847 | Tryptophol | HMDB0003447 |
| 6.7 | 437.2666 | 138.9796 | LysoPA (18:0/0:0) | HMDB0007854 |
| 6.7 | 426.0114 | 44.9976/133.0136 | Adenosine phosphosulfate | HMDB0001003 |
| 8.1 | 482.3262 | 91.0542/65.0386 | LysoPE (18:0/0:0) | HMDB0011130 |
| 8.3 | 965.5226 | 67.0183/210.0878 | PIP (22:5(4Z, 7Z, 10Z, 13Z, 16Z)/16:0) | HMDB0010029 |
| 9.4 | 411.2483 | 69.0340/167.0708 | LysoPA (16:0/0:0) | HMDB0007853 |
| 9.5 | 87.0432 | 43.0542/341.3050 | Diacetyl | HMDB0003407 |
| 10.2 | 279.2303 | 711.2905/659.3167 | Gamma-Linolenic acid | HMDB0003073 |
| 10.8 | 319.2276 | 43.0547/73.0289 | 12-KETE | HMDB0013633 |
| 11.1 | 758.6021 | 261.2576 | PE (22:0/P-16:0) | HMDB0009510 |
| 11.2 | 319.2276 | 163.1117 | 5,6-Epoxy-8,11,14-eicosatrienoic acid | HMDB0002190 |
| 11.7 | 756.5477 | 60.0807/712.4911 | PC (14:0/20:3(8Z, 11Z, 14Z)) | HMDB0007882 |
| 11.7 | 139.1110 | 72.0807/80.9736 | (3Z, 6Z)-3,6-Nonadienal | HMDB0031152 |
| 11.8 | 647.5075 | 144.0801 | SM(d18:1/12:0) | HMDB0012096 |
| 11.9 | 401.3395 | 45.0340/51.0234 | 7a-Hydroxy-cholestene-3-one | HMDB0001993 |
| 12.0 | 343.1892 | 189.1643 | 11-Dehydrocorticosterone | HMDB0004029 |
| 12.0 | 729.5899 | 175.1117 | SM(d18:0/18:1(11Z)) | HMDB0012088 |
| 12.1 | 717.5526 | 105.0704 | SM (00d18:0/16:1(9Z) (OH)) | HMDB0013463 |
| 12.5 | 291.2302 | 464.4462 | 4-Methylphenyl dodecanoate | HMDB0037711 |
| 12.7 | 393.3003 | 69.0704/311.2374 | Murocholic acid | HMDB0000811 |
| 12.8 | 349.2379 | 631.4445 | Tetrahydrocorticosterone | HMDB0000268 |
| 13.0 | 756.5477 | 135.1173 | PC (20:3(8Z, 11Z, 14Z)/14:0) | HMDB0008394 |
| 13.3 | 377.2344 | 69.0698/161.1324 | 14-Hydroxy-E4-neuroprostane | HMDB0012580 |

TABLE 2
The detailed information of differential metabolites in serum samples.

| RT (min) | Precursor MS | MS/MS | Compound name | HMDB ID |
|---|---|---|---|---|
| 1.6 | 177.1035 | 160.0746 | Serotonin | HMDB0000259 |
| 2.0 | 221.0923 | 155.0609 | 5-Hydroxy-L-tryptophan | HMDB0000472 |
| 3.7 | 477.2479 | 105.0704 | Retinoyl b-glucuronide | HMDB0003141 |
| 4.8 | 817.7204 | 619.4809 | SM(d18:0/24:0) | HMDB0012094 |
| 5.4 | 835.7694 | 852.802 | TG (16:0/16:0/18:0) | HMDB0005357 |
| 6.3 | 714.5378 | 86.0964/574.3867 | PC (14:1(9Z)/P-18:1(9Z)) | HMDB0007931 |
| 11.1 | 754.5362 | 72.0807/696.4598 | PC (20:4(8Z, 11Z, 14Z, 17Z)/14:0) | HMDB0008459 |
| 11.2 | 548.3700 | 75.0440/365.3050 | LysoPC (20:2(11Z, 14Z)/0:0) | HMDB0010392 |
| 11.5 | 297.2416 | 153.1273 | 9,10-Epoxyoctadecenoic acid | HMDB0004701 |
| 12.0 | 758.5691 | 617.5508 | PE (22:2(13Z, 16Z)/15:0) | HMDB0009549 |
| 12.2 | 717.5548 | 659.4758 | SM (d18:0/16:1(9Z) (OH)) | HMDB0013463 |
| 12.5 | 317.2470 | 91.0542 | Alpha-dihydroprogesterone | HMDB0003069 |
| 12.7 | 736.4876 | 285.2429 | PE (22:6(4Z, 7Z, 10Z, 13Z, 16Z, 19Z)/14:0) | HMDB0009679 |
| 12.7 | 550.3858 | 184.0733 | PC (18:1(9Z) e/2:0) | HMDB0011148 |
| 15.4 | 951.7379 | 881.6654 | TG (20:4(5Z, 8Z, 11Z, 14Z)/ | HMDB0005478 |
| | | | 20:4(5Z, 8Z, 11Z, 14Z) | |

To further investigate the metabolic pathways of CDD-2103 in DSS mice, we imported these differential metabolites to MetaboAnalyst 4.0. As shown in FIGS. 3C and 3D, based on pathway impact>0.1, three pathways were affected significantly in the feces and serum, namely tryptophan metabolism, linoleic acid metabolism, and glycerophospholipid metabolism. The metabolites related to these pathways were choline, PC (16:0/16:0), arachidonic acid, linoleic acid, L-tryptophan, methoxyindoleacetate, phosphatidylserine, phosphatidylcholine, and 12,13-EpOME.

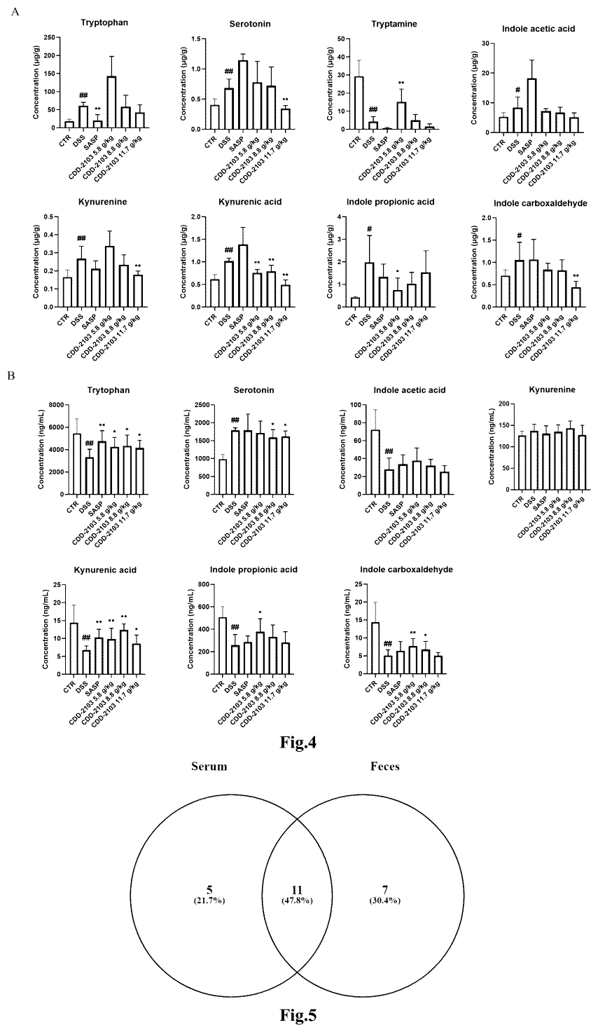

Studies have revealed that tryptophan metabolites are involved in immune regulation by modulating intestinal microbiota composition and homeostasis. Our untargeted metabolomics study showed the significant changes in the tryptophan metabolites profiles, demonstrating the important role of tryptophan in the pathogenetic development of UC. Therefore, we carried out targeted quantification analysis of the metabolites involved in tryptophan metabolism (FIGS. 4A and 4B). In the fecal samples, except for tryptamine (TM), all tryptophan metabolites were significantly up-regulated in the DSS group compared with the CTR group. All these metabolic disorders were reversed to some extent after treatment with CDD-2103, whilst SASP only showed its therapeutic effect on tryptophan. In addition, we suspect that kynurenic acid (KA) was a key metabolite that CDD-2103 was targeting, because all dosages of CDD-2103 significantly reduced the up-regulated levels of KA caused by DSS treatment. In the serum samples, there was no significant differences in kynurenine (KYN) level among different groups, and TM was undetectable. For other metabolites, except for serotonin (5-HT), all tryptophan metabolites were significantly down-regulated in the DSS group compared with the CTR group, while CDD-2103 could reverse all these conditions. Combined fecal and serum results of DSS-treated mice indicated that, CDD-2103 had a therapeutic effect on KA, 5-HT, and indole carboxaldehyde (IC) levels. Among these, KA might be the most critical metabolite that CDD-2103 exhibited the anti-colitis effect in DSS-induced chronic colitis mice by regulating tryptophan metabolism.

### 2.4 Serum and Feces Component Analysis of CDD-2103

The peaks detected in the biological samples and not in the blank sample were interpreted as corresponding to the absorbed or dis-absorbed compounds in the intestinal. A total of 14 prototypes and 2 metabolites were identified in rat serum, and 15 prototypes compounds and 3 metabolites were identified in rat feces; 11 prototypes were detected in both serum and feces (FIG. 5). The compounds of CDD-2103 identified in the rat serum and feces are summarized in Table 3.

TABLE 3
Compounds of CDD-2103 in rat serum and feces identified by UPLC-Q-TOF/MS.

| | | | | Positive ion mode | | | |
|---|---|---|---|---|---|---|---|
| | | | | | | Mass | |
| | RT | | | | | accuracy | MS/MS |
| No | (min) | Formula | Compound | Adduct | m/z | (ppm) | (fragments) |
| 1 | 3.56 | C₁₇H₂₆O₁₁ | Morroniside* | [M + Na]⁺ | 429.1358 | −2.1 | 385/267 |
| 2 | 5.64 | C₁₇H₂₆O₁₀ | Loganin* | / | / | / | / |
| 3 | 6.00 | C₂₀H₂₄NO₄⁺ | Magnoflorine* | [M]⁺ | 342.1696 | −2.63 | 297/265/237 |
| 4 | 6.44 | C₈H₁₄O₅ | α-Isobutylmalic acid | / | / | / | / |
| 5 | 6.48 | C₆H₁₂O | Gamma-Hexenol | / | / | / | / |
| 6 | 8.91 | C₂₆H₂₈NO₁₀⁺ | Jatrorrhizine-3-O- | [M]⁺ | 514.1706 | −1.36 | 485/434/417 |
| | | | beta-D-glucuronide | | | | |
| 7 | 9.86 | C₂₁H₂₂O₉ | Liquiritin* | / | / | / | / |
| 8 | 11.16 | C₁₉H₁₆NO₄⁺ | Berberrubine* | [M]⁺ | 322.1069 | −3.1 | 307/279 |
| 9 | 11.85 | C₆H₁₂ | Methylcyclopentane | / | / | / | / |
| 10 | 14.25 | C₁₉H₁₄NO₄⁺ | Coptisine* | [M]⁺ | 320.0914 | −2.81 | 305/292/277 |
| 11 | 14.31 | C₂₀H₂₀NO₄⁺ | Columbamine* | [M]⁺ | 338.1382 | −2.96 | 323/294 |
| 12 | 14.43 | C₂₀H₁₈NO₄⁺ | Epiberberine* | [M]⁺ | 336.1229 | −2.08 | 320/292 |
| 13 | 14.78 | C₂₀H₂₀NO₄⁺ | Jatrorrhizine* | [M]⁺ | 338.1384 | −2.37 | 323/294 |
| 14 | 18.05 | C₂₀H₁₈NO₄⁺ | Berberine* | [M]⁺ | 336.1229 | −2.08 | 321/292 |
| 15 | 18.05 | C₂₁H₂₂NO₄⁺ | Palmatine* | [M]⁺ | 352.1542 | −1.99 | 337/322/308 |
| 16 | 18.07 | C₃₄H₄₆O₁₇ | Militarine* | / | / | / | / |
| 17 | 19.06 | C₂₀H₁₆NO₇ | Cop+ 3hydroxylation+ | [M]⁺ | 382.092 | 1.83 | 364/345/318/297 |
| | | | methylation | | | | |
| 18 | 21.02 | C₄₂H₆₂O₁₆ | Glycyrrhizic acid* | [M + H]⁺ | 823.4091 | −2.43 | 648/472/454 |
| 19 | 22.30 | C₁₉H₁₆O₄ | Bisdemethoxycurcumin* | [M + H]⁺ | 309.1121 | 0.00 | 225/189/147 |
| 20 | 22.54 | C₁₅H₂₀O₃ | Atractylenolide III* | [M + H]⁺ | 249.1486 | −2.02 | 229/203/187 |
| 21 | 22.60 | C₂₀H₁₈O₅ | Demethoxycurcumin* | [M + H]⁺ | 339.1223 | −1.18 | 255/177/147 |
| 22 | 22.86 | C₂₁H₂₀O₆ | Curcumin* | [M + H]⁺ | 369.1335 | 0.54 | 285/177 |
| 23 | 25.02 | C₃₂H₄₈O₆ | Poricoic acid DM | / | / | / | / |

| | Negative ion mode | | | | | | | |
|---|---|---|---|---|---|---|---|---|
| | | | Mass | | | | | |
| | | | accuracy | MS/MS | In | In | | |
| No | Adduct | m/z | (ppm) | (fragments) | serum | feces | Type | Origin |
| 1 | [M + COOH]⁻ | 451.1462 | 1.11 | 405/243/141 | + | / | P | SY |
| 2 | [M + COOH]⁻ | 435.1491 | −3.91 | 227/101 | + | / | P | SY |
| 3 | / | / | / | / | + | + | P | HL |
| 4 | [M − H]⁻ | 189.0772 | 2.12 | 171/145/129 | + | + | P | BJ |
| 5 | [M + COOH]⁻ | 145.0872 | 1.38 | 134/119/103 | / | + | M | JS |
| 6 | / | / | / | / | + | / | M | HL |
| 7 | [M − H]⁻ | 417.1195 | 0.96 | 255/135 | + | / | P | GC |
| 8 | / | / | / | / | + | + | P | HL |
| 9 | [M + COOH]⁻ | 129.0927 | 4.65 | 116/101 | / | + | M | GC |
| 10 | / | / | / | / | + | + | P | HL |
| 11 | / | / | / | / | + | + | P | HL |
| 12 | / | / | / | / | + | + | P | HL |
| 13 | / | / | / | / | + | + | P | HL |
| 14 | / | / | / | / | + | + | P | HL |
| 15 | / | / | / | / | + | + | P | HL |
| 16 | [M + COOH]⁻ | 771.2700 | −2.72 | 726/457 | + | + | P | BJ |
| 17 | / | / | / | / | + | / | M | HL |
| 18 | / | / | / | / | + | + | P | GC |
| 19 | [M − H]⁻ | 307.0966 | −3.26 | 187/143/119 | / | + | P | JH |
| 20 | [M − H]⁻ | 247.1336 | −1.62 | 203/187 | / | + | P | BZ |
| 21 | [M − H]⁻ | 337.1077 | −1.19 | 217/173/119 | / | + | P | JH |
| 22 | [M − H]⁻ | 367.1186 | −0.27 | 217/173/149 | / | + | P | JH |
| 23 | [M − H]⁻ | 527.3373 | −0.95 | 485/467/423 | / | + | M | FL |

“*” represents the compounds determined by comparison with reference standards;
“P” represents prototype, and “M” represents metabolite;
SY: wine-processed Corni Fructus;
HL: Coptidis Rhizoma;
BJ: Bletillae Rhizoma;
JS: Paederiae Scandentis Herba et Radix;
GC: honey-processed Glycyrrhizae Radix et Rhizoma;
JH: Curcumae Longae Rhizoma;
BZ: bran-processed Atractylodis Macrocephalae Rhizoma;
FL: Poria

### 2.5 Network Pharmacology

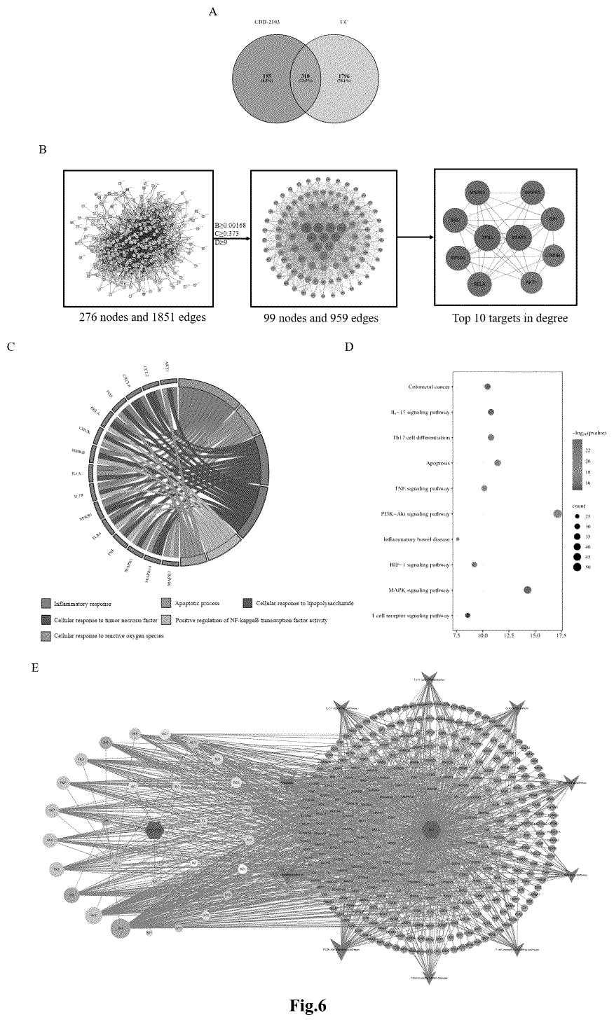

23 compounds, including prototypes and metabolites, identified in the serum-feces pharmacochemistry study were subjected to network pharmacology study. According to HERB and TTD database, a total of 505 targets were derived from these compounds. From GeneCards, a total of 2106 genes related to UC were obtained; of these, 310 overlapped with the CDD-2103 targets in the Venn diagram analysis (FIG. 6A). Then, the 310 intersection targets were subjected to STRING database. Targets with a confidence value>0.9 were screened out, and independent nodes were deleted; the remaining were imported to Cytoscape for network construction. Using the Network Analyzer tool, 99 core nodes whose betweenness, closeness, and degree greater or equal to the median (≥0.00168, ≥0.373, ≥9, respectively) were screened out. The top 10 genes with higher degrees were considered critical genes; these were TP53, STAT3, MAPK3, SRC, EP300, MAPK1, RELA, JUN, CTNNB1, and AKTI (FIG. 6B).

To identify the functions and mechanisms of CDD-2103 in UC remission, GO and KEGG pathway enrichment analyses were carried out based on the 310 overlapping targets. For GO-term analysis, CDD-2103 against UC mainly involved in apoptosis, inflammatory response, and positive regulation of NF-kappaB transcription factor activity (FIG. 6C). KEGG enrichment analysis further revealed that CDD-2103 targeted proteins were enriched in colorectal cancer, inflammatory bowel disease, IL-17, and MAPK signaling pathways (FIG. 6D), which were correlated with the pathological mechanisms of UC.

To further elucidate the relationship between herbs, compounds, genes, pathways, and diseases, an herb-compound-gene-pathway-disease network was constructed. This comprised 7 herbs, 21 compounds, 310 targets, and 10 pathways from the above prediction (FIG. 6E). Among these, the top 5 compounds from different herbs were curcumin, berberine, liquirtin, militarine, and poricoic acid DM; the top 5 targets with the greatest number of edges were PTGS2, NOS2, ESR1, MAPK14, and GSK3B; and pathways enriched by more genes were the PI3K-Akt and MAPK signaling pathways.

### 2.6 Integrated Analysis of Metabolomics and Network Pharmacology

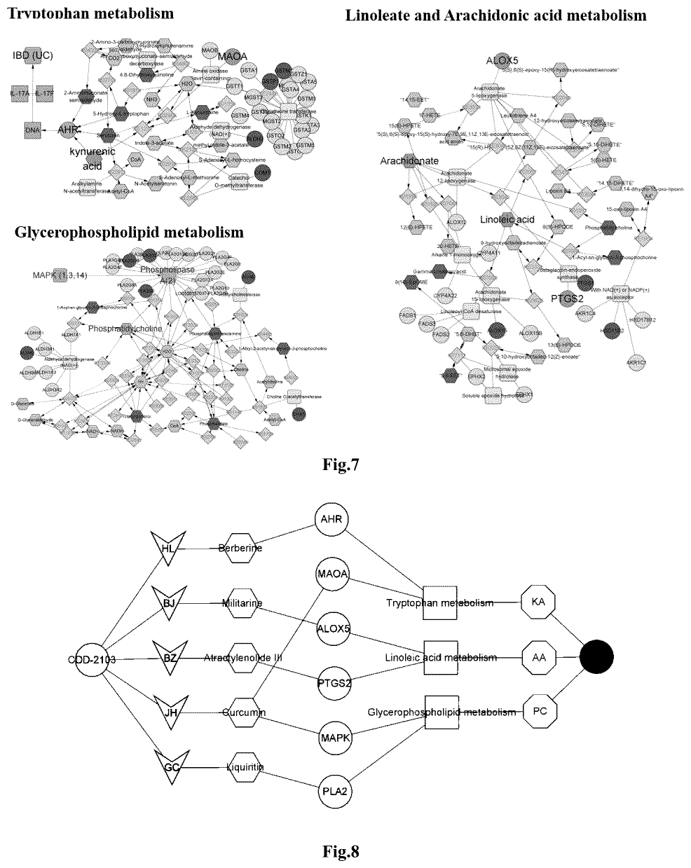

To obtain a comprehensive view of the mechanisms of CDD-2103 against UC, we constructed two integrated interaction networks based on metabolomics analysis and network pharmacology and matched them by Cytoscape. First, based on the differential metabolites and the potential targets, the compound-reaction-enzyme-gene network showed that MA0A, AHR, MAPK, PLA2, ALOX5, and PTGS2 might be the key targets, and that, KA, phosphatidylcholine (PC), and arachidonic acid (AA) might be the key metabolites regulated by CDD-2103 (FIG. 7). Second, based on the herb-compound-target-pathway-metabolite-disease network, we found that tryptophan, glycerophospholipid and linoleic acid metabolisms might be the key metabolic pathways, and that, curcumin, berberine, atractylenolide III, liquiritin, and militarine might be the key active ingredients (FIG. 8). Finally, matching the two comprehensive networks revealed how these metabolites, targets, pathways, and ingredients might interact in the therapeutic effect of CDD-2103 on UC. They are summarized in Table 4 and await further validation.

TABLE 4
Key active ingredients, targets, metabolites, and pathways
for CDD-2103 in UC remission as revealed by
integrated analysis of metabolomics and network pharmacology.

| Pathway | Metabolite | Target | Active compound |
|---|---|---|---|
| Tryptophan | KA | MAOA, | Curcumin, berberine |
| metabolism | | AHR | |
| Glycerophospholipid | PC | PLA2, | Liquiritin, curcumin |
| metabolism | | MAPK | |
| Linoleic acid | AA | PTGS2, | Atractylenoide |
| metabolism | | ALOX5 | III, militarine |

KA: kynurenic acid;
PC: phosphatidylcholine;
AA: arachidonic acid

### 2.7 Molecular Docking

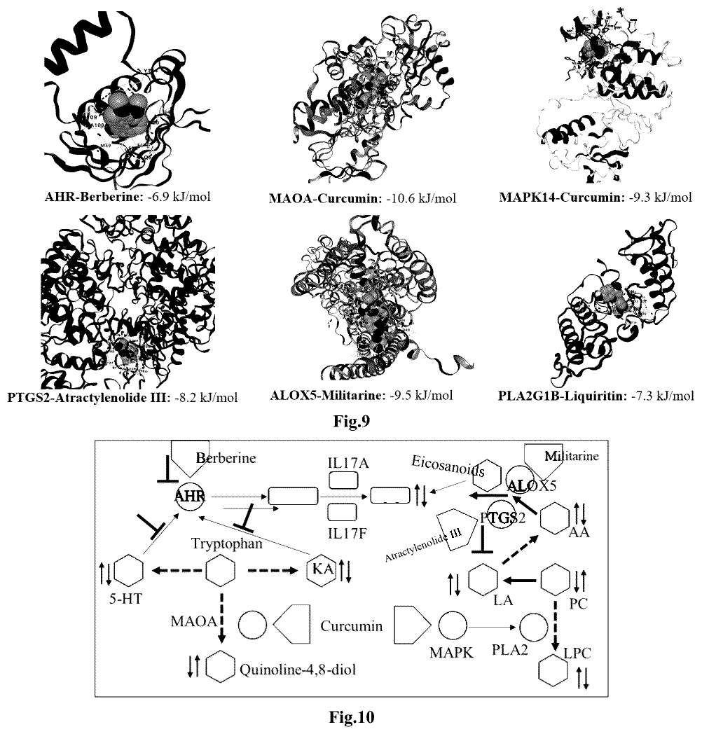

To further investigate the possibility of interaction between CDD-2103 and the key targets, we performed molecular docking studies (FIG. 9). After searching the RCSB Protein Data Bank database, we found six key targets that could be analyzed by molecular docking. The docking analysis of AHR showed that berberine had strong affinity −6.9 kJ/mol and the hydrogen-bonding interaction sites were SER24, THR52, ASN181, PHE352, and ALA448. The interactions with MA0A showed that curcumin had the strongest affinity −10.6 kJ/mol, and the interaction sites were SER24, TYR69, ILE180, ASN181, GLN215, ALA272, PHE352, and ALA448. In addition, curcumin showed very strong affinity with MAPK14 (−9.3 kJ/mol), and the interaction sites were PRO191, TYR200, TYR258, LEU262, ASP292, and ASP294. In the interactions with PTGS2, atractylenolide III had strong affinity as well and the hydrogen-bonding interaction sites were CYS1036, ASN1039, GLU1046, CYS1047, GLN1461, and LYS1468. For ALOX5, militarine showed strong affinity and the binding sties were ARG37, ASP40, ASN102, HIS617, and VAL633. For PLA2G1B, the docking results showed that liquiritin had hydrogen-bonding interactions with ASN24, TYR28, GLY33, LEU118, and THR120 and the energy was −7.3 kJ/mol. These docking results indicated the high affinities among the herbal compounds of CDD-2103 and the key targets, especially MA0A and ALOX5.

### 3. Discussion

UC is a chronic relapsing gastrointestinal disease that is difficult to treat. Although several anti-inflammatory drugs are applied in UC patients, these medications cannot successfully maintain long-lasting remission in UC patients. This demonstrates the urgent need to discover a new medication for UC rescue. TCM formulas are generally composed of combined herbs and compounds; according to traditional principles and practice, multiple herbs increase efficacy, counteract side effects, and moderate effects. It is generally believed, biochemically, that these activities are achieved via multiple targets and pathways. This study found that a TCM formulation CDD-2103 could significantly attenuate ulcerous symptoms and colonic injuries in the DSS-induced chronic colitis mice model. We used a novel strategy, involving metabolomics, network pharmacology, serum-feces pharmacochemistry and molecular docking to identify its active compounds, regulating targets, and pathways that are involved in its effect on UC.

In the present work, 23 compounds, including phototypes and metabolites, were identified in the serum and feces of rats after CDD-2103 administration. We understood these as active components exhibiting biological functions via either entering the circulation or modulating the gut microbiota. These identified compounds were subjected to network pharmacology for target prediction. By integrating metabolomics and serum-feces pharmacochemistry-based network pharmacology, we uncovered that the five major active compounds (curcumin, berberine, atractylenolide III, liquiritin, and militarine) from the herbs strongly bind to six key targets (MAOA and MAPK14, AHR, PTGS2, PLA2G1B, and ALOX5, respectively), regulating related metabolic pathways (tryptophan metabolism, glycerophospholipid metabolism, and linoleic acid metabolism) to alleviate UC, and that the key metabolites (KA, PC, and AA) can serve as biomarkers for diagnosis and treatment. These findings provide a specific biochemical framework for understanding the functional nature of CDD-2103 in UC remission (FIG. 10).

Tryptophan metabolites comprise host-derived metabolites, such as kynurenic acid (KA) from the kynurenine pathway, and serotonin (5-HT) from the 5-HT pathway, as well as bacteria-derived metabolites, including indole derivatives and tryptamine (TM) from the indole pathway. Most of the tryptophan metabolites can bind with aryl hydrocarbon receptor (AhR) to aggravate or prevent the inflammatory responses in IBD. For instance, on the one hand, KA activates G protein-coupled receptor 35 (GPR35) in the bowel wall, which may aggravate DSS-induced chronic inflammation by affecting nod-like receptor protein 3 (NLRP3). On the other hand, KA also plays an anti-inflammatory role by regulating oxidative stress and inhibiting LPS-induced pro-inflammatory cytokines, such as IL-1 and IL-6. 5-HT, a key metabolite from the 5-HT pathway, can also alter numerous immune cells and modulates the gut microbiota composition through binding with AHR. Suppression of 5-HT production in the intestine has been proven to reduce inflammation in DSS-treated mice. Indole carboxaldehyde (IC), derived from the gut bacterial metabolism of tryptophan, leads AHR-dependent IL-22 production and mucosal protection. Studies showed that serum IC was decreased in DSS-induced acute colitis mice. In the current study, the up-regulated levels of both KA and IC in DSS-treated mice were reversed by CDD-2103 treatment in the serum samples, although KA and IC levels in fecal samples of DSS mice were down-regulated, CDD-2103 could also reverse its metabolic perturbation. Moreover, the up-regulated 5-HT levels in the serum and fecal samples of DSS-induced colitis mice could also be reversed by CDD-2103. Therefore, CDD-2103 comprehensively exhibited its therapeutic effect on kynurenine, 5-HT, and indole pathways of tryptophan metabolism, and KA, 5-HT, and IC could be understood as the corresponding metabolites that CDD-2103 targeted in both feces and serum samples. Based on network analysis, berberine, and curcumin predicted the binding activity with AHR, and it was verified by molecular docking. This might be another mechanism by which CDD-2103 prevents the inflammation of DSS-treated mice by regulating tryptophan metabolism.

Lipids are in cell membranes, where they are responsible for mucus production, barrier integrity, and intra- and intercellular signaling. Phosphatidylcholine (PC), a metabolite of dietary choline, is the major bioactive phospholipid for mucus composition. The increasing activity of phospholipase A2 (PLA2), one of the degradation enzymes of PC, leading to the production of lysophosphatidylcholine (LPC) and arachidonic acid (AA), is deduced as the cause of PC content decreasing in UC patients. LPC exhibits proinflammatory effects via binding with dendritic cells to produce IFN-γ, resulting in greater tissue destruction. AA is the essential bioactive molecule enriched in linoleic acid metabolism and can be metabolized to pro-inflammatory eicosanoids such as prostaglandin E2 (PGE2). Phosphatidylethanolamine (PE) is a key constituent of lipid rafts. Mitogen-activated protein kinase (MAPK) is discovered in lipid rafts to sense the inflammatory cytokines such as TNF-α and IL-lip, then mediate inflammatory responses. In the present study, CDD-2103 significantly increased PC and PE levels in the serum samples, while the increasing level of LPC was reversed by CDD-2103 in the fecal samples of DSS-treated mice. The binding effect of liquirtin on PLA2G1B (the first member of the PLA2 family) might be the possible mechanism by which CDD-2103 adjusted the LPC/PC ratio to alleviate the disease severity. Moreover, the MAPK signaling pathway was enriched in network pharmacology studies, and MAPK14 was predicted as the hub gene. The biological function of PE related to this pathway, alkaloids from HL, and curcuminoids from JH could be considered as the active compounds from CDD-2103 acting on this part. These results supported the possible hypothesis that CDD-2103 protects intestinal mucosa by regulating glycerophospholipid metabolism.

Inflammation in IBD is correlated with a high level of production of eicosanoids from linoleic acid metabolism. Metabolites from this metabolic pathway are mainly composed of ω-6 fatty acid, and arachidonic acid (AA); the metabolite of linolenic acid (LA), is the primary precursor for eicosanoids' production. In the early stage of inflammation, the expression of ALOX5 and PTGS2 increases, leading to the production of PGE2, and other eicosanoids from AA. PGE2 can act as an irritant in the gut lumen, to relax the colonic muscles and contribute to diarrhea. Furthermore, MA0A (a metabolic enzyme of 5-HT and TM), is an intestinal stem cell marker that was inhibited by PGE2 following TNF stimulation in intestinal organoids. 5-aminosalicylic compounds, such as SASP used in the current study as the positive drug, can suppress PGE2 levels to exhibit the anti-colitis effect in a dose-dependent manner. In our study, LA and AA were increased in the serum samples of the DSS-treated mice, whilst CDD-2103 could reverse this condition. These findings revealed that CDD-2103 compounds, including atractylenolide III, and militarine might exhibit its anti-colitis effect by regulating ALOX5 and PTGS2, to inhibit the production of eicosanoids derived from linoleic acid metabolism.

It can be seen from the above description that, based on the comprehensive analysis of metabolomics and network pharmacology based on serum-feces pharmacochemistry, this application finds that several active compounds in CDD-2103 can regulate MA0A, AHR, MAPK14, PLA2G1B, ALOX5 and PTGS2, and regulate the metabolism of tryptophan, glycerolphospholipid and linoleic acid. This comprehensive strategy has been proven to have a powerful role in effectively discovering active compounds and mechanisms of action in traditional Chinese medicine prescriptions. Therefore, the UC medical model and medical method based on CDD-2103 provided in this application can effectively prevent and treat UC by regulating tryptophan metabolism, glycerolphospholipid metabolism and linoleic acid metabolism by establishing a strong combination between the active ingredient group of CDD-2103 and the key target combination of UC. This application also discloses the use of CDD-2103 as a biomarker for the diagnosis and treatment of UC, which can be used for the diagnosis and monitoring of UC prevention and treatment.

However, the active ingredient group disclosed in this application that can effectively treat UC may not only come from CDD-2103, but may also come from one or more other drug formulations. Thus, as long as the drug contains both the active ingredient group claimed for patent protection in this application, and the active ingredient group can establish a strong connection with the UC key target combination disclosed in this application, thereby preventing and treating UC by regulating tryptophan metabolism, glycerolphospholipid metabolism, and linoleic acid metabolism. All fall within the scope of protection of this application.

The above provides a detailed explanation of the technical solution and effects of the present application in conjunction with the accompanying drawings and specific embodiments in the specification. It should be noted that the specific embodiments disclosed in the specification are only the preferred embodiments of the present application, and technical personnel in the field can also develop other embodiments based on this; Any simple deformation and equivalent replacement that does not deviate from the innovative concept of this application are covered by this application and fall within the scope of protection of this patent.

What is claimed is:

1. An active ingredient group for treating UC, characterized by:
the active ingredient group includes curcumin, berberine, atractylolactone III, glycyrrhizin and militarine.

2. The active ingredient group for treating UC according to claim 1, characterized in that:
the active ingredient group can act on the key target combination of UC, which includes MA0A, MAPK14, AHR, PTGS2, PLA2G1B, and ALOX5.

3. The active ingredient group for treating UC according to claim 1, characterized in that:
there is a strong binding between the active ingredient group and the key target combination of UC.

4. The active ingredient group for treating UC according to claim 1, characterized in that the active ingredient group can regulate tryptophan metabolism, glycerolphospholipid metabolism, and linoleic acid metabolism.

5. The active ingredient group for treating UC according to claim 1, characterized in that the biomarkers for treating UC using the active ingredient group are KA, PC, and AA.

6. The active ingredient group for treating UC according to claim 1, characterized in that the active ingredient group comes from CDD-2103.

7. The active ingredient group for treating UC according to claim 6, characterized in that the CDD-2103 comprises:
Codonopsis Radix,
Bran-processed Atractylodis Macrocephalae Rhizoma,
Poria,
Wine-processed Corni Fructus,
Coptidis Rhizoma,
Curcumae Longae Rhizoma,
Paederiae Scandentis Herba et Radix,
Bletillae Rhizoma,
Honey-processed Radix Glycyrrhizae.
